(19) United States
(12) Patent Application Publication
(10) Pub. No.: US 2023/0187024 A1
(43) Pub. Date: Jun. 15, 2023

(54) Method and device for decoding data stored in a DNA-based storage system

(71) Applicant: Université de Bretagne Sud, LORIENT (FR)

(72) Inventor: Laura CONDE-CANENCIA, LORIENT (FR)

(21) Appl. No.: 17/925,562

(22) PCT Filed: May 11, 2021
(86) PCT No.: PCT/FR2021/050826

§ 371 Date: Nov. 15, 2022

(30) Foreign Application Priority Data
May 15, 2020 (FR) FR2004819

(51) Int. Cl. G16B 30/20; G16B 40/10; G16B 50/00; G06F 30/20; H03M 13/11; H03M 13/00

(52) U.S. Cl. CPC G16B 30/20; G16B 40/10; G16B 50/00; G06F 30/20; H03M 13/1111; H03M 13/611; G06F 2111/08

(57) ABSTRACT

A method includes obtaining, for each type of nucleotide, a probability density function, the probability density functions being obtained from measurements of current drops produced during at least one passage of at least one sequence of reference nucleotides through a nanopore sequencer; obtaining measurements of current drops produced when the sequence of nucleotides to be decoded passes through the nanopore sequencer; calculating, for each measurement value considered and for each type of nucleotide of the B types of nucleotides, a piece of reliability information based on the probability density function obtained for the type of nucleotide considered; obtaining a decoded value identifying a type of nucleotide from the B types of DNA nucleotides, by applying a soft decoding algorithm with an error correction code to the current drop measurement and to the B pieces of reliability information obtained for the considered measurement value.

### TECHNICAL FIELD

The present description relates to a device for decoding data stored in a DNA (deoxyribonucleic acid)-based storage system with a nanopore sequencer and a corresponding decoding method.

### STATE OF THE ART

One method of reading data stored in DNA-based storage systems is known as DNA sequencing. Their goal is to determine the exact nucleotides and their order in a DNA sequence that encodes digital data.

There have already been several generations of sequencing technologies and each of them has raised specific challenges. After first-generation Sanger sequencing dating back to the 1970s, second-generation technologies [25] have enabled an impressive decrease in sequencing costs over the past decade (for example, [26] and [27]). However, these technologies cannot read long strands of nucleotides: the devices must thus read short fragments and then combine the data to recover the original sequence. This process has motivated recent research on reconstruction algorithms ([28], [29] and [17]).

In this document, only the so-called third generation sequencers that are based on nanopores are considered. In these sequencers (also called sequencing devices here), the principle of nanopore sequencing is based on the detection of changes in an ionic current when a DNA sequence passes through a nanoscale hole. Each nucleobase or nucleotide causes a different amplitude of current drop due to its different atomic structure. This makes it possible to identify the nucleotide passing through the nanopore at a given time. The main advantage of nanopore sequencers is that they make it possible to read long sequences in a single step, up to several tens of thousands of nucleotides.

However, these nanopore sequencers still have significant limitations and a high error rate. Thus, the challenge is to provide effective tools to correct errors inherent in the sequencing technology, which correspond to single or burst deletions, insertions and substitutions that may occur during sequencing.

The process of synthesizing DNA sequences encoding digital data is also known to be a source of single substitution and deletion/insertion errors.

These most promising chemical DNA synthesis methods are known as micro-array based synthesis. These methods make it possible to synthesize DNA sequences up to 200 nucleotides in length, at a cost of about $0.001 per nucleotide. However, its major drawback is its high error rate. From a general point of view, we can say that the current synthesis methods combine either a high cost and high precision, or contrariwise, a low cost and low precision, and the current research is aimed at reducing the gap between these two extremes.

The main error-generating events in DNA synthesis are single substitutions [1] [2] [10] of one nucleotide for another, and the substitution error rates depend mainly on the performance and cost of the technology [13] [14] [15].

Sequencing methods using Polymerase Chain Reaction (PCR) amplify these substitution errors by creating numerous copies of the synthesized sequence. In addition, with high-throughput sequencing, synthesis errors propagate through a number of reads produced by the sequencing. These issues have been addressed in [17] [18] [19] with the introduction of DNA profile codes.

Thus, there is a need to improve the situation by reducing the complexity of DNA-based systems using nanopore-based sequencers while increasing their reliability, in particular by reducing the substitution errors produced during sequencing.

In this perspective, previous works include [8] [14] [15]. In these approaches, the authors propose asymmetric codes to deal with substitution errors characterized by the statistical distributions (probability density functions of the output signal levels) of the impulse responses of the output signals of the nanopore sequencer, these output signals corresponding to the amplitudes of the measured current drops. These errors are actually considered asymmetric because some substitutions are much more likely than others (for example. nucleobase A is more likely to be substituted by nucleobase T than by nucleobase G).

In [15], codes in the Damerau distance are introduced to correct individual or block transposition errors combined with deletions. Other significant work [16] addresses the problem of rapid translocation rates of DNA molecules through the nanopore, which leads to burst deletions [17]. To correct this type of errors, the authors proposed a non-binary code for correcting burst deletions in [18]. All these kinds of codes offer firm error correction capability and the associated decoding algorithms use bounding distance decoding, based on making a hard decoding decision at the input of the decoder.

### SUMMARY

According to a first aspect, there is disclosed a method for decoding a sequence of binary data encoded by a sequence of nucleotides to be decoded comprising B types of DNA nucleotides, B being an integer equal to 2, 3 or 4, the decoding method comprising
obtaining, for each type of nucleotide of the B types of nucleotides, a probability density function, the probability density functions being obtained from measurements of current drops produced during at least one passage of at least one sequence of reference nucleotides through a nanopore sequencer;
obtaining measurements of current drops (y₁, y₂, . . . yₖ) produced when the sequence of nucleotides to be decoded passes through the nanopore sequencer;
calculating, for at least one measurement value and for each type of nucleotide among the B types of nucleotides, a piece of reliability information (λ^{k}(i)^{L}) based on the probability density function obtained for the type of nucleotide considered;
obtaining for each considered measurement value a decoded value identifying a type of nucleotide from the B types of DNA nucleotides by applying a soft decoding algorithm with an error correction code to the current drop measurement and to the B pieces of reliability information obtained for the considered measurement value.

In example embodiments, the probability density function is a Gaussian probability density function and the soft decoding algorithm is based on modeling the current drop measurement produced by the nanopore sequencer, a noisy variable modulated by pulse-amplitude modulation with discrete levels, each level corresponding to an average value of the probability density function obtained for a given type of nucleotide, the modulated noisy variable being made noisy by B channels of additive white Gaussian noise corresponding respectively to the statistical distributions obtained for the B types of nucleotides. The nanopore sequencer is thus modeled as an asymmetric communication channel.

In example embodiments, the error correction code is a turbo code or an LDPC, Low-Density Parity-Check code decoding algorithm. The soft decoding algorithm is for example a turbo-code algorithm of MAP, Message Parsing Algorithm, type. The soft decoding algorithm is for example a Min-Sum algorithm for LDPC codes or a belief propagation (BP) algorithm for LDPC codes.

In example embodiments, the number B of nucleotide types is equal to 4 and the soft decoding algorithm with an error correction code is applied to symbols in a Galois Field of order 4, with each symbol in the Galois Field of order 4 corresponding to a nucleotide. The method is also applicable to RNA (ribonucleic acid) sequences comprising only 3 types of nucleotides while using a Galois body of order q=4. The order in which the nucleotides are associated with the symbols in the Galois Field of order 4 corresponds to the inverse order of the average values of the probability density functions of the current drop amplitudes obtained for the different nucleotide types.

In example embodiments, the reliability information for a measurement value yₖ and a nucleotide type i is calculated as follows:

$${{λ}^{k} ( i )}^{L} \frac{{( {y}_{k} - {C}_{i} )}^{2}}{2 {σ}_{i}^{2}} \qquad [ Math \text{.201} ]$$

where Ci is the mean value of the probability density function and σi is the standard deviation of the probability density function obtained for nucleotide type i.

According to a second aspect, a decoding device is disclosed comprising means for implementing the steps of a method according to the first aspect. The means may be hardware and/or software means configured to implement the functions defined in this document for the decoding device. According to an example embodiment, the decoding device comprising at least one memory and at least one processor, the memory storing program instructions configured to cause said decoding device to execute the steps of a method according to the first aspect when the program instructions are executed by the processor.

According to yet another aspect, there is disclosed a computer program comprising program instructions for executing the steps of a method according to the first aspect when said program is executed by a computer.

According to yet another aspect, there is disclosed a computer-readable recording medium on which is recorded a computer program comprising program instructions for executing the steps of a method according to the first aspect when said program is executed by a computer.

According to yet another aspect, a DNA-based data storage system is disclosed, comprising a nanopore sequencer and a decoding device according to the second aspect.

### BRIEF DESCRIPTION OF THE FIGURES

Further advantages and particularities will become apparent from the following description, given as a non-limiting example and made with reference to the attached figures in which:

FIG. 1 shows aspects of parity block calculation in an encoding method according to one or several embodiments;

FIG. 2 is a schematic representation of a DNA-based storage system according to one or several embodiments;

FIG. 3 shows examples of statistical distributions of measurements of current drops obtained for different types of nucleotides in a nanopore sequencer;

FIG. 4 shows a table of parameters (mean and standard deviation) of the statistical distributions presented in FIG. 3;

FIG. 5 shows a flowchart of a soft decoding method according to one or several embodiments.

### DETAILED DESCRIPTION

DNA-based data storage systems with nanopore sequencers will now be described in more detail. These storage systems are based on the use of quaternary codes based on graphs defined on Galois Fields of order 4 and associated decoding algorithms based on “soft samples”. These samples are the products of nanopore sequencers whose main characteristic is the passage of the DNA sequence at controlled speed through one or more nanopores. In these systems, each DNA nucleotide is represented as an element of a Galois Field of order 4. Likelihood calculations are introduced to take into account an asymmetric DNA channel model and the ionic current drops. A Min-Sum algorithm adapted for low complexity quasi-optimal decoding is presented. The simulation results show that the error correction method proposed in this document is able to guarantee a data read that is nearly free from substitution errors and has ideal conditions for synthesis.

The term “soft decoding” or soft samples is used here to refer to a decoding technique in which reliability information is available for the samples. This reliability information makes it possible, especially, to correct the value of the sample measured at a given time. A soft sample or soft value is an analog value measured at the output, without quantization or coding.

The contributions described in this document specifically address the correction of substitution errors produced during nanopore sequencing.

The first contribution relates to the use of a quaternary encoding/decoding scheme using a representation of the DNA nucleotides as elements of a Galois Field of order 4 (this also includes matching them to the numerical information to be stored in the system) and the use of non-binary correction codes defined in this Galois Field. This make it possible to avoid the quaternary-to-binary conversions that would be required when using a binary encoding/decoding scheme.

The second contribution relates to the use of statistical distributions of ion current signal amplitudes as “soft” intrinsic information, that is, as reliability information. Likelihood calculations at the graph-based decoder that take into account these amplitudes and the specific model of asymmetric DNA channel without memory (i.e. the output at a given time depends only on the input at that time, but not on the inputs at previous or later times). Note that the coding approaches known in the prior art [8] [15] for this problem do not exploit the “soft” information provided by the nanopore system. Error correction codes that can use this intrinsic information can be LDPC (Low-Density Parity-Check) codes or Turbo codes.

Finally, as one of the major drawbacks of non-binary symbol decoding algorithms is their complexity, a third contribution consists of adapting the Min-Sum algorithm to be able to perform quasi-optimal decoding with a reduced number of calculations.

### Definitions and Principles

As a reminder, a Galois field GF(q) is a finite set of q elements whose every element can be described in terms of a primitive element, denoted here as a. The elements (or symbols) of the Galois field GF(q) are denoted {0, α⁰, α¹, . . . α^{q-2}}.

A codeword is denoted by X={x₁, x₂, . . . . x_{N}}, where{xₖ}, k=1 to N, is an element (or symbol) belonging to a Galois field GF(q) and is represented by m=log₂(q) bits, with xₖ={x_{k,1}, x_{k,2}, . . . , x_{k,m}}.

LPDC codes are error correction codes of the linear block code category, whose low-density parity check matrix has the property of being “hollow”, that is it contains only a small number of non-zero elements compared to its total number of elements. LDPC codes can indeed be characterized, like linear block codes, by what is known as a parity matrix, generally denoted H. The parity matrix H is linked to what is known as a code generation matrix, generally denoted G by the relationship: G. H^{T}=0 where H^{T} is the transposed matrix of H. The dimensions M×N of the parity matrix correspond, for the number of rows M, to the number of parity constraints of the code, and for the number of columns N, to the length of the codewords of the considered code (i.e. the number of symbols in a codeword). The rows of the parity matrix H of a linear block code correspond respectively to parity constraints which are by design fulfilled by the codewords, the equation v. H^{T}=0 will be correct for any codeword v.

LDPC codes whose symbols composing the codewords belong to the binary Galois field (of order 2), denoted GF(2), are said to be binary, whereas the LDPC codes whose symbols composing the codewords belong to a Galois field of order q strictly higher than 2, denoted GF(q), are said to be non-binary. Thus, the elements of a parity matrix of a non-binary LDPC code will belong to a non-binary Galois field GF(q) (q>2) and the matrix products of the above equations will be performed using the addition and multiplication laws of the GF(q) field, denoted respectively ⊕ and ⊗ hereinunder.

A non-binary LDPC code is a linear code associated with an input data block and defined by a very sparse parity check matrix H whose non-zero elements belong to a finite field GF(q), where q>2. In this document, we consider the case of a Galois field of order q=4.

The construction of these LDPC codes is expressed as a set of parity check equations in GF(q), where a parity equation involving d_{c} codeword symbols is written:

Σₖ₌₁^{d}^{_{c}}h_{j,k}xₖ=0 [Math. 1]

where hj_{,k} are the non-zero values of the j-th row of the matrix H and a codeword is denoted by X={x₁, x₂, . . . . x_{N}}, where xₖ is a symbol represented by m=log₂(q)=2 bits in the case where q=4. We denote as d_{c} the number of 1's in a row of the matrix H and dᵥ the number of 1's in a column of the matrix H.

Many algorithms for iterative decoding of LDPC codes use a representation of the parity matrix of the code by a bi-partite graph called a “Tanner graph”. For a parity matrix H of dimensions M×N, this representation uses branches to match M nodes, called “Check Nodes” (CN), with N nodes, called “Variable Nodes” (VN). Each non-zero element of the parity matrix is represented in the corresponding Tanner graph by a branch joining the check node corresponding to the row of the element in the matrix H to the variable node corresponding to the column of the element in the matrix H. Each check node in the graph thus represents a parity equation determined by the branches connecting it to the variable nodes.

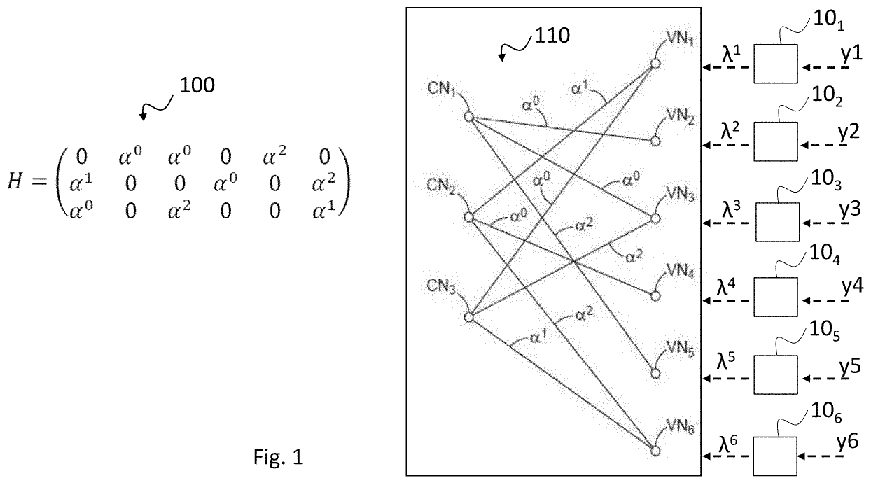

An example of a parity check matrix H 100 is shown in the left part of FIG. 1 and the corresponding Tanner graph 110 is shown in the right part of FIG. 1. In this example, the parity matrix H is defined in the Galois field GF(4) whose elements are denoted {0, α⁰, α¹, α²}. The dimensions of the parity matrix H are M=3 and N=6. The corresponding Tanner graph 110 comprises M=3 check nodes denoted in FIG. 1 CN₁, CN₂ and CN₃, and N=6 variable nodes denoted VN₁, VN₂, VN₃, VN₄, VN₅ and VN₆.

The three parity equations corresponding respectively to the three check nodes of this graph are:

α¹⊗VN₂⊕α⁰⊗VN₃⊕α²⊗VN₅=0 [Math. 11]

for the check node CN₁,

α¹⊗VN₁⊕α⁰⊗VN₄⊕α²⊗VN₆=0 [Math. 12]

for the check node CN₂,

α⁰⊗VN₁⊕α²⊗VN₃⊕α¹⊗VN₆=0 [Math. 13]

for the check node CN₃,
where the operators ⊕ and ⊗ respectively denote addition and multiplication in the Galois field GF(4).

The Tanner graph 110 represents the iterative processing applied to each codeword to be decoded. The variable nodes VNₖ each receive a vector λ^{k} composed of B=4 pairs of values for each input measurement value yₖ corresponding to a symbol of an input codeword to be decoded or a codeword being decoded. For example, in FIG. 1, the variable node VNₖ receives a vector A^{k} computed by a computational block 10ₖ from the input yₖ to decode the symbol xₖ of a codeword X.

These pieces of reliability information are for example based on the LLR (Log Likehood Ratio) function as explained in more detail in this document. In this example, each pair in the vector λ^{k} is composed of a possible symbol xₖ in GF(4)={0, α⁰, α¹, α²} and an associated reliability information, denoted λ^{k}(i)^{L} with i=1 to 4. The input vector λ^{k} is thus defined as follows:

$${λ}^{k} = ( \begin{matrix}{{λ}^{k} ( 1 )}^{L} & 0 \\ {{λ}^{k} ( 2 )}^{L} & {α}^{0} \\ {{λ}^{k} ( 3 )}^{L} & {α}^{1} \\ {{λ}^{k} ( 4 )}^{L} & {α}^{2}\end{matrix} ) \qquad [ Math . 14 ]$$

The decoded codeword is generated by the variable nodes after iteration(s) in the Tanner graph. A decoded symbol of a decoded codeword corresponds to the first value in the output vector (the symbols in the output vector being sorted in ascending order of LLR value, so that the first row of the output vector comprises the highest LLR value and the associated decoded symbol), that is the value in GF(4) that is most likely or has the lowest decoding error. The variable node VNₖ thus receives a decoded vector containing the decoded value for the symbol xₖ of the codeword X. The decoded symbol identifies one type of nucleotide among the B types of DNA nucleotides and corresponds to the current drop measurement value.

The Tanner graph representation can be used to implement decoding algorithms whose efficiency has been shown on graph models, such as the belief propagation (BP) algorithm or message passing (MP) algorithms. When applied to a bipartite graph with two types of nodes, the BP algorithm relies on an iterative process of sending messages between nodes of each type connected by branches (so-called “neighbor nodes”).

In the context of an LDPC code decoding algorithm, a message corresponds to a data vector. A message can be an intrinsic message (an input data vector generated from the channel information at the decoder input) or an extrinsic message (a data vector generated during an iteration applied to an intrinsic message, these extrinsic messages are the messages exchanged between the check nodes and the variable nodes).

Iterative LDPC code decoding algorithms based on Tanner graph, using especially the exchange of messages between the check nodes and the variable nodes of the Tanner graph corresponding to the LPDC code considered, thus have been developed. These decoding algorithms can more generally be implemented or adapted for decoding all linear block codes that can be represented by a bipartite graph comprising a set of check nodes and a set of variable nodes.

The Tanner graph of a non-binary LDPC code is generally much sparser than a corresponding Tanner graph of a binary LDPC code with the same bit rate and code length [19] [20]. In addition, better error correction performance can be achieved by using the lowest possible degree of variable node, that is dᵥ=2.

For DNA data storage, we consider in this document non-binary LDPC codes defined on a Galois Field of order 4 denoted GF(4) (that is q=4). The elements of GF(4) are thus denoted {0, α⁰, α¹, α²}, where a is the primitive element of this Galois Field. An element of GF(q) is thus represented by m=log₂(q)=2 bits. The elements of GF(4) are also called symbols.

The basic building blocks of DNA are the four nucleotides: Adenine (A), Cytosine (C), Guanine (G) and Thymine (T). Each nucleotide is represented by a symbol in GF(4). Prior to synthesis, the input binary data sequence is converted to a quaternary data or symbol sequence in GF(4).

The quaternary data sequence is then divided into blocks of K=N−M input symbols, corresponding respectively to the nucleotides A, C, G and T, to result in input codewords of size N. After LDPC coding, a codeword C with redundancy is obtained for each input codeword, the codeword C with redundancy comprising N symbols in GF(4) and consisting, on the one hand, of a block of K symbols in GF(4) corresponding respectively to the K input symbols and, on the other hand, of a parity check block of M symbols in GF(4) calculated on the basis of the coefficients of the parity matrix. The LDPC coding is repeated for each input codeword or block so as to obtain a succession of codewords with redundancy.

A succession of codewords with redundancy is then synthesized so as to obtain a DNA sequence encoding the input binary data blocks.

A nanopore device is then used to convert the DNA sequence encoding the input binary data blocks into a sequence of voltage drop amplitude measurements. This sequence of current drop amplitude measurements is converted into a sequence of symbols in GF(4) by applying a soft decoding algorithm (soft input decoding algorithm), by associating to each current drop amplitude measurement q=4 reliability values corresponding respectively to the 4 symbols of GF(4). As an output of the decoding algorithm, a symbol xₖ decoded in GF(4) is obtained for each current drop amplitude measurement yₖ. The decoded symbol corresponds to the likeliest symbol, the one for which the decoding error is the lowest.

The soft decoding algorithm can be an LDPC code decoding algorithm (for example, a Min-Sum algorithm for LDPC codes or a BP, for Belief Propagation, algorithm) or turbo codes (for example, a MAP, Message Parsing Algorithm). As part of this document, the use of an LDPC algorithm is described in more detail. Whatever the decoding algorithm, the reliability values used are based on the statistical distributions of measurements of current drops produced when a reference sequence composed of nucleotides of a given type (excluding other types of nucleotides) passes through a nanopore sequencer. For example, if soft-input LDPC decoding is applied, the exchanged messages comprise the symbols of the processed codewords and a piece of reliability information associated with each symbol.

In example embodiments, pieces of reliability information are calculated from a measurement value yₖ provided by the nanopore device. These pieces of reliability information are based on the LLR (Log Likehood Ratio) function as explained in more detail in this document.

It will be noted that when the LDPC code is binary and the symbols of the codewords are with values in the Galois field GF(2), that is with binary values, the exchanged messages comprise probability densities comprise two densities, one for the value “0” and the other for the value “1”. The messages or data vectors comprise for example pairs of binary values with which likelihood (or reliability) values are respectively associated.

In the context of this document, the LDPC code considered is non-binary, and the symbols of the codewords have values in the Galois field GF(4). The exchanged messages contain q=4 reliability values, each corresponding to an element of GF(q), which can be represented as a vector of size q of pairs (symbol, reliability value).

A check node of a decoder for non-binary LDPC codes of a Galois field-valued code GF(q) thus receives d_{c} input messages and generates d_{c} messages as output. Each input and output message contains q=4 pairs of values, one representing a symbol, and the other representing a reliability or likelihood associated with that symbol.

When using a direct implementation of the Belief Propagation (BP) decoding algorithm, an output is constructed by selecting the best q combinations among q to the power d_{c}−1. This leads to a computational complexity on the order of O(q²). The BP decoding algorithm can also be considered in the frequency domain. This is then called a Fourier Transform-based BP algorithm. Moving into the frequency domain makes it possible to reduce the complexity of the BP algorithm, in order to reach a complexity of the order of O(d_{c}×q×log(q)). However, the implementation of the BP algorithm has a very high cost in terms of computational complexity, a cost that becomes prohibitive as soon as values of q greater than 16 are considered.

Different algorithms have been proposed to overcome this high complexity problem, among which the so-called Extended Min-Sum (EMS) algorithm, which proposes to use truncated messages by selecting the most reliable nₘ symbols, nₘ being chosen to be much lower than q (nₘ<<q). However, given the relatively low order of the Galois field, we consider here that nₘ=q=4.

The messages are then sorted before being fed to the check node. A check node can be formed by a combination of elementary check nodes, where each elementary check node receives as input two sorted messages each containing nₘ pairs (symbol, reliability) from which it generates an output message containing the nₘ best possible combinations of the two input messages, the total number of combinations being equal to nₘ to the power of 2.

Please refer to the following article for a detailed description of the EMS algorithm: “Decoding algorithms for nonbinary LDPC codes over GF(q),” D. Declercq and M. Fossorier, IEEE Trans. Commun., vol. 55, no. 4, pp. 633-643, April 2007.

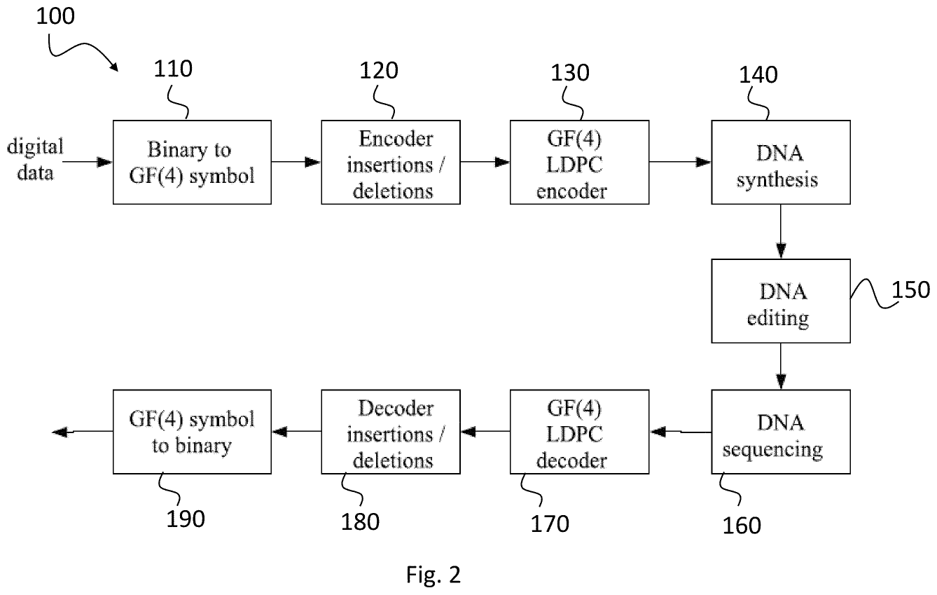

FIG. 2 shows a simplified model of a chain of components 110 to 190 of a DNA-based storage system 100. Note that this model does not include a component for compression.

This chain of components comprises:
a component 110 for converting binary data into GF(4) symbols;
a component 120 for correcting insertion/deletion errors;
an LDPC encoding component 130 applied to GF(4) symbols;
a writing component 140, that is of DNA synthesis;
a DNA editing component 150;
a reading component 160, that is of DNA sequencing;
an LDPC decoding component 170 applied to GF(4) symbols;
a component 180 for correcting insertion/deletion errors;
a component 190 for converting GF(4) symbols into binary data.

Component 110 is configured to convert binary data into GF(4) symbols. Component 110 implements a function for converting between binary data and GF(4) symbols defined as follows:

‘00’->0
‘01->α⁰
‘10’->α¹
‘11’->α².

Symmetrically, component 190 is configured to convert GF(4) symbols into binary data and uses the conversion function inverse to that of component 110.

Component 120 is configured to introduce, during coding, correction codes for insertion/deletion errors. Symmetrically, component 180 is configured to use, during decoding, error correction codes to correct insertion or deletion errors introduced at the time of encoding.

In this document, we focus specifically on substitution errors, that is, we aim to isolate the problem of substitutions occurring during nanopore sequencing. Thus, components 120 and 180 are complementary to components 130 and 170, which aim at correcting substitution errors.

Component 130, called here the GF(4) encoder, implements functions for encoding the data blocks coming from component 120 involving the generation of parity check blocks, by means of error correction codes of the LDPC (Low-Density Parity-Check) or turbo code type, defined on a Galois field of order 4, so that each encoded symbol corresponds to one of the four basic nucleotides of the DNA (that is ‘A’, ‘T’, ‘C’ and ‘G’).

LDPC coding generates parity check blocks. This component 130 is applied to a succession of elements in the Galois field GF(4). Symmetrically, component 170, referred to here as the LDPC GF(4) decoder, uses the parity check codes to correct errors on the output data blocks of component 160. These components will be described in more detail below.

More precisely, component 130 is configured to encode a sequence of quaternary data (or symbols in GF(4)) using LDPC or turbo coding. For example, in the case of LDPC coding, the sequence of symbols is divided into blocks of K=N−M input symbols to result in input codewords of size N. After LDPC coding, a codeword C with redundancy is obtained for each input codeword, the codeword C with redundancy comprising N symbols in GF(4) and consisting, on the one hand, of a block of K symbols in GF(4) corresponding respectively to the K input symbols and, on the other hand, of a parity check block of M symbols in GF(4) calculated on the basis of the coefficients of the parity matrix. The LDPC coding is repeated for each input codeword or block so as to obtain a succession of codewords with redundancy that will be processed by the DNA synthesis component 140.

Component 140 is configured to perform DNA synthesis from the incoming symbol string in GF(4). The synthesis is based on the following matching function:

### 0->A

### α⁰->T

### α¹->C

a²->G.

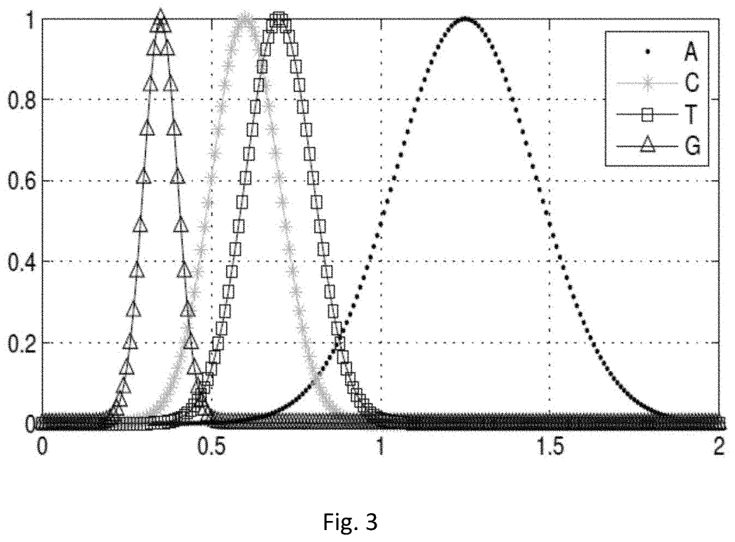

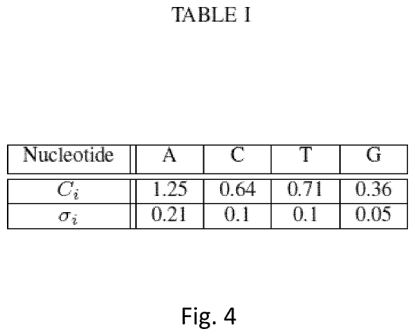

As will be explained in more detail below, the order in which nucleotides are considered and associated with the symbols 0, α, α¹, α² in GF(4) corresponds to the inverse order of the average values (highest to lowest) of the probability density functions of the current drop amplitudes obtained for a given nucleotide. In the example of FIG. 3 and as specified in the table of FIG. 4, the nucleotide A has the highest mean value, followed in order by the nucleotides T, C then G.

Simple insertion/deletion error corrections can also be used by integrating correction codes during synthesis, that is, at the component 140 level. The Tenengolts codes [cf: G. Tenengolts Nonbinary Codes “Correcting Single Deletion or Insertion” IEEE Transactions on Information Theory vol. IT-30 pp. 766-769 1984] are well adapted to this type of errors and can be directly encoded in the DNA sequence. In addition, the problem of DNA sequence reconstruction from deletion/insertions followed by PCR techniques has been discussed in [9] [10]. Since we focus in this document specifically on nanopore sequencing techniques, we assume that sequence reconstruction is ideal or that we do not have to worry about it. Note, however, that sequence reconstruction has motivated a great deal of recent research [10] [11]. In addition, components 120 and 180 will not be described further in this document.

Symmetrically to component 140, component 160 is configured to perform DNA sequencing via a nanopore sequencer and thus read a DNA sequence.

Component 150 is configured for DNA editing and corresponds to a process of deletion and insertion of DNA substrings at well-controlled locations. In addition, editing can be performed by adding very specific point mutations [24] [25]. These possibilities will not be described further in this document.

In order to make the DNA-based storage system more robust, data channel coding principles are exploited at the decoding component 170 and adapted to the sequencing operation. In particular, the operation of measuring the current drop amplitude during nanopore sequencing (component 160) is modeled as a data transmission channel so that decoding can benefit from reliability information to perform soft decoding associated with the measurement values, that is of measured current drop amplitudes for each nucleotide.

Thus, the current drop amplitude measurements obtained by component 160 are converted to symbols in GF(4) by means of a soft decoding process using reliability information.

During the soft decoding performed by component 170, the parity check blocks generated by encoding component 130 are exploited, are integrated into the soft decoding (for N inputs, there are M symbols corresponding to the parity check blocks) and thus make it possible to correct the substitution errors during sequencing.

The work presented in [26] shows that it is possible to optimize the translocation rates for single nucleotides passing through a nanopore. This is possible due to the high viscosity of some ionic liquids at room temperature. The current drops have been statistically characterized and show that substitution errors become dominant. Thus, the soft decoding method presented in this document is particularly suitable for correcting errors produced by sequencing. Nanopore sequencing (component 160) generates output measurement values of the current drops produced by the passage of the DNA sequence through the nanopore are transmitted to component 170.

Details of the Soft Decoding Method (Component 170)

For modeling purposes, each measurement value of a current drop corresponds to a sample. A sample corresponds to the realization of a Gaussian random variable.

FIG. 3 represents the statistical distributions obtained for 4 types of nucleotides ‘A’, ‘T’, ‘C’ or ‘G’ respectively. Each curve represents a statistical distribution of the current drop values obtained for one type of nucleotide. In practice, since the DNA chain cannot be formed with only one type of nucleotide, several known DNA chains are used passing through a nanopore sequencer, and the current drop values are measured many times (1000, 2000, etc.) to obtain the statistical distribution for a given type of nucleotide. In this case, each statistical distribution corresponds to a Gaussian probability density function represented by a Gaussian curve corresponding to a Gaussian random variable.

For example, in FIG. 3:
nucleotide ‘A’ is associated with a Gaussian curve whose mean corresponds to a current drop of 1.25 nA;
nucleotide ‘T’ is associated with a Gaussian curve whose mean corresponds to a current drop of 0.68 nA;
nucleotide ‘C’ is associated with a Gaussian curve whose mean corresponds to a current drop of 0.65 nA;
nucleotide ‘G is associated with a Gaussian curve whose mean corresponds to a current drop of 0.3 nA.

Assuming that the four nucleotides are equiprobable, a hard decoding process that would use thresholds to identify each nucleotide would lead to high error rates because the curves are highly overlapping. For example, as shown in FIG. 3, when the value of the current drop is between 0.3 and 1 it is not possible to know for sure or with sufficient probability whether it is the G, C or T nucleotide. On the other hand, if the value of the current drop is higher than 1, the probability that it is nucleotide A is practically 100%.

To significantly reduce these error rates, a soft decoding algorithm is used. More precisely, we use an extended Min-Sum algorithm [27] applied to elements in a Galois Field of order q=4. This algorithm is based on a generalization of the Min-Sum algorithm used for binary LDPC codes presented in [28] [29].

The current drop measured at a given time is modeled as a variable modulated by 4-level pulse amplitude modulation (here denoted 4-PAM), each level corresponding to the average value of the probability density function of the current drop amplitudes obtained for a given nucleotide. Moreover, the modeling takes into account these statistical distributions by adding to this modulated variable 4 channels of additive white Gaussian noise corresponding respectively to the statistical distributions obtained for the 4 nucleotides.

Calculation of the Intrinsic Message

Considering 4-PAM modulation and B=4 channels of additive white Gaussian noise (“AWGN”), the noisy current samples received at the output of the nanopore sequencer constitute a noisy sequence Y of N symbols in GF(4) independently affected by a noise, where each sample is denoted yₖ=PAM(xₖ)+nₖ, k=1 to B. The modulation coefficient is represented by PAM(xₖ) and nₖ is a random variable that follows a Gaussian probability density function with zero mean and variance denoted

σᵢ² [Math. 2]

with i=1 to 4. The value of the standard deviation a depends on the type of nucleotide A, G, C, T causing the present current drop and is determined from the normalized probability density function of the current drops for each DNA nucleotide, according to, for example, the values given in the table shown in FIG. 4. In this table, we give the mean values C₁ to C₄ and the standard deviations σ₁ to σ₄ for the 4 distributions corresponding to the 4 nucleotides. This table is an example of possible values for a given sequencing sequencer. In practice, for each sequencer and for given experimental conditions, a statistical analysis is implemented for this sequencer, in order to obtain the mean values and standard deviations specific to the sequencer used and/or the experimental conditions.

Let L^{k}(X), k=1 to N, be the value of the log likelihood ratio (LLR) of the k-th symbol xₖ representing sample k, k=1 to N, in an N-symbol codeword X and being the symbol GF(4) that maximizes the probability of yₖ knowing x, denoted P(yₖ|x) (conditional probability). The first step of the Min-Sum algorithm is the calculation of the value L^{k}(x) for each symbol x of the codeword. With the assumption that the four nucleotides are equiprobable, the value L^{k}(x) of the symbol x in the codeword can be defined by:

$${L}^{k} ( x ) = ln ( \frac{P ( {y}_{k} \vert {\tilde{x}}_{k} )}{P ( {y}_{k} \vert {x}_{k} )} ) \qquad [ Math \text{.4} ]$$

$$with$$

$${\tilde{x}}_{k} = arg \begin{matrix}max \\ {x}_{∈} G F ( 4 )\end{matrix} \{ P ( {y}_{k} \vert x ) \} \qquad [ Math \text{.5} ]$$

It should be noted that

L^{k}(\tilde{x}ₖ)=0 [Math. 6]

and that for any symbol x of GF(4)

Lₖ(x)≥0 [Math. 7]

Thus, as the value of L^{k}(x) of a symbol x increases, its reliability decreases. This definition of L^{k}(x) avoids having to re-normalize messages after each node update.

Let λ^{k} be the intrinsic Min-Sum message associated with the k-th symbol xₖ knowing yₖ. The intrinsic message λ^{k} is a vector composed of 4 pairs (λ^{k}(i)^{L}, λ^{k}(i)^{GF}) pour i=1 to 4, where λ^{k}(i)^{GF} is a symbol GF(4) (λ^{k}(1)^{GF}=0, λ^{k}(1)^{GF}=α, λ^{k}(1)^{GF}=α¹, λ^{k}(1)^{GF}=α²) et Δ^{k}(i)^{L} (is the associated LLR value computed based on the Math 8 formula below and such that: λ^{k}(i)^{L}=L^{k}(λ^{k}(i)^{GF}). The LLR values λ^{k}(i)^{L} for i=1 to 4 satisfy the relationship: λ^{k} (1)^{L}≤λ^{k} (2)^{L}≤λ^{k} (3)^{L}≤λ^{k}(4)^{L}.

With the asymmetric channel model considered, the variable yₖ is considered a noisy variable modulated by pulse-amplitude modulation with B=4 discrete levels, each level corresponding to an average value of the probability density function of the measured current drop measurements for a given nucleotide among the B nucleotide types. It is possible to calculate λ^{k}(i)^{L} as follows:

$${{λ}^{k} ( i )}^{L} = \frac{{( {y}_{k} - {C}_{i} )}^{2}}{2 {σ}_{i}^{2}} \qquad [ Math \text{.8} ]$$

where Ci is the mean value of the probability density function and σi is the standard deviation of the probability density function obtained for nucleotide type i. Cᵢ and σᵢ result from a statistical analysis specific to the sequencer used, such as for example the values presented in the table in FIG. 4. Note that for i=λ^{k}(i)^{L}=0.

The intrinsic message at the input of the decoding algorithm is formed by 4 pairs consisting of: an LLR λ^{k}(i)^{L} value and a symbol in GF(4), and they are ordered according to the LLR value obtained by equation Math. 8. The LLR values λ^{k}(i)^{L} are normalized, starting with 0, according to the equation Math. 8. given above. The soft decoding algorithm is thus based on modeling the current drop measurement produced by the nanopore sequencer as a noisy variable modulated by pulse-amplitude modulation with B discrete levels, each level corresponding to an average value of the Gaussian probability density function of the measurements of current drops measured for a given nucleotide from the B types of nucleotides, the modulated noisy variable being made noisy by B channels of additive white Gaussian noise corresponding respectively to the statistical Gaussian distributions obtained for the B types of nucleotides.

Definition of Messages Corresponding to Edges of the Tanner Graph

We define here two types of extrinsic messages or data vectors corresponding to the edges of the Tanner graph: for an edge of this graph, we define a C2V (“Check to Variable”) message going from the check node CN to the variable node VN and a V2C (“Variable to Check”) message going from the variable node VN to the check node CN. For the edge corresponding to the element h_{j,k}, we denote as C2V(j,k) and V2C(j,k) the associated messages.

Since dᵥ=2, only 2 edges are connected to a given variable node VN k. We denote as C2V(jₖ(1),k) and C2V(jₖ(2),k) (respectively V2C(jₖ(1),k) and V2C(jₖ(2),k)) the two C2V (respectively V2C) messages associated with the VN k where jₖ(1) and jₖ(2) indicate the position of the two non-zero values of column k of the matrix H.

Similarly, we denote as C2V(j, kⱼ(v)) (respectively V200, kⱼ(v))) for v=1 to d_{c}, the d_{c} C2V (respectively V2C) messages associated with CH j where kⱼ(v) indicate the position of the v-th non-zero value of row j of the matrix H.

The structure of the V2C and C2V messages is identical to the structure of the intrinsic message λ^{k}. The V2C output message of a VN must contain only the 4 sorted LLR values V2C(I)^{L} and the associated GF symbols V2C(I)^{GF}, with I=1 to 4. Similarly, the C2N output message from CN contains the 4 LLR values C2N(I)^{L} (sorted in ascending order) and their associated GF symbols C2N(I)^{GF}.

Treatment for Each VN

For a symbol x, L(x), V2C(x) and C2V(x) are respectively the intrinsic LLR values, the extrinsic V2C and C2V messages associated with the symbol x. The VN decoding equations can be divided into three steps.

Step 1: the calculation of V2C(x) for each x in GF(4)

V2C(x)=C2V(x)+L(x) [Math. 11]

Step 2: Determining the minimum value of V2C)

$$\tilde{x} = arg \begin{matrix}min \\ {x}_{∈} G F ( 4 )\end{matrix} \{ V 2 C ( x ) \} \qquad [ Math \text{.12} ]$$

Step 3: Standardization

V2C(x)=V2C(x)−V2C(\hat{x}) [Math. 13]

Treatment for Each CN

A check node of degree d_{c} can be broken down into elementary check nodes, for example into 3(d_{c}−2) elementary check nodes.

To minimize the computational complexity, it is possible to use the Bubble-check algorithm at the elementary check node level described for example in E. Boutillon, L. Conde-Canencia, “Simplified check node processing in nonbinary LDPC decoders”, 6th International Symposium on Turbo Codes & Iterative Information Processing, Brest, France, September 2010.

Decoding by a Min-Sum Algorithm

A parity check matrix H is obtained. The non-zero values of H can be chosen randomly from the elements of GF(4).

In an example embodiment, ultra-sparse (very low density) GF(4)-LDPC codes that are based on the protograph [21] [22] with dᵥ=2 are used. The corresponding matrices are designed to maximize the circumference of the associated bipartite graph, and minimize the multiplicity of cycles with minimum length [23]. Each parity check block uses exactly dᵥ=2 distinct symbols in GF(4). This limitation in the choice of values reduces storage requirements.

The Min-Sum algorithm is applied on the basis of the Tanner bipartite graph associated with the parity check matrix H obtained on this basis.

The extended Min-Sum algorithm is applied here using new equations at the first stage of the decoder (that is, the intrinsic log-likelihood-ratio calculations). Furthermore, since we aim to use low-complexity check node processing, we adapt the prior work to the case of hardware-friendly GF(4)-LDPC implementations.

The decoding process iterates nᵢₜ times and for each iteration the following operations are performed: M updates of check nodes CN (M being the number of check nodes) and M*dc updates of variable nodes VN. In the last iteration, a decision is made for each symbol, the decoded GF(4) symbols are then generated and constitute the decoded DNA codeword.

The decision for the codeword is made in the VN processors and concludes the decoding process. The decoder is then applied to the next code mode.

Steps of Decoding by a Min-Sum Algorithm

The steps of the Min-Sum algorithm can be summarized as follows.

In an initialization phase, the intrinsic message is generated for k=1 to N:<

{L^{k}(x)}_{x∈GF}(4) [Math. 20]

this intrinsic message corresponding to the 4 values L^{k}(x) calculated on the basis of the formula Math. 1.

The V2C message is calculated for k=1 to N and v=1 to 2:

V2Cⱼ_{ₖ}₍ᵥ₎^{k}=L^{k} [Math. 21]

The soft decoding is then performed in an iterative way. At each iteration, the following steps are implemented. The number of iterations is fixed. For j=1 to M: we perform steps A) B) and C).

A) calculation of the extrinsic message V2C associated with the check node CNⱼ for v=1 to d_{c}:

V2Cⱼ^{k}^{ⱼ}^{(v)} [Math. 22]

B) implementation of the processing associated with the check node CNⱼ for v=1 to d_{c} in order to generate new C2V messages

C2Vⱼ^{k}^{ⱼ}^{(v)} [Math. 25]

C) for each variable node kⱼ(v) connected to the check node CNⱼ, we update the second message V2C using the new C2V message and the intrinsic message L^{k}

V2Cⱼ^{k}^{ⱼ}^{(v)} [Math. 23]

Then steps A) to C) are repeated in the next iteration.

At the end of the iterations, a final decision is made to estimate the codeword using the new C2V message and the intrinsic L^{k} message.

The decision êₖ for k=1 to N is expressed as the symbol x on GF(4) that minimizes the sum below:

$${\hat{e}}_{k} = arg \begin{matrix}min \\ {x}_{∈} G F ( 4 )\end{matrix} \{ C 2 {V}_{{j}_{k} ( 1 )}^{k} ( x ) + C 2 {V}_{{j}_{k} ( 2 )}^{k} ( x ) + {L}^{x} ( x ) \} \qquad [ Math \text{.24} ]$$

For more details on this Min-Sum algorithm, one can refer to the document by Boutillon, E., Conde-Canencia, L., Al Ghouwayel, A. “Design of a GF(64)-LDPC Decoder Based on the EMS Algorithm” IEEE Transactions on Circuits and Systems I, vol. 60, no. 10, pp. 2644-2656, October 2013, doi: 10.1109/TCSI.2013.2279186 2013.

Other detailed examples of Min-Sum algorithms are described in references [29] and [28].

Decoding Method

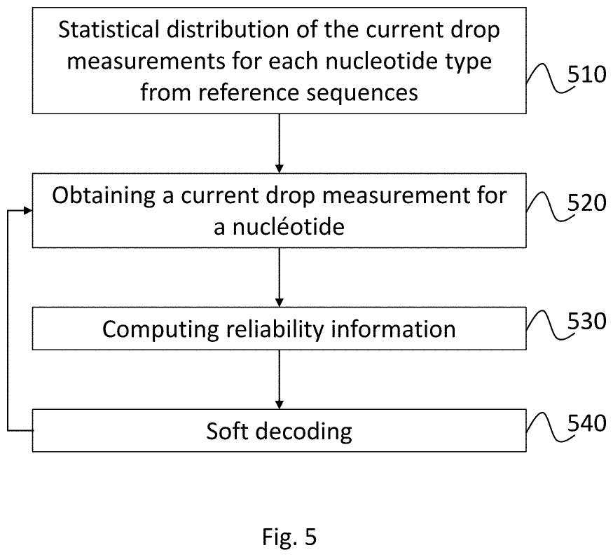

FIG. 5 shows a schematic diagram of a soft decoding method. The method is applied to a binary data sequence encoded by a sequence of nucleotides to be decoded comprising B types of nucleotides, for example the B=4 nucleotides Adenine (A), Cytosine (C), Guanine (G) and Thymine (T). The method is also applicable to RNA (ribonucleic acid) sequences comprising only 3 types of nucleotides while using a Galois body of order q=4.

Although the steps are described sequentially, the steps can be performed in a different order and/or in parallel. Some steps may be repeated or omitted. The characteristics and aspects of data processing described in this document, particularly with reference to FIGS. 1 to 4, are applicable to the implementation of this method.

In a step 510, for each type of nucleotide among the N types of nucleotides, a Gaussian probability density function of measurements of current drops is obtained for each type of nucleotide among the B types of nucleotides. These probability density functions are obtained from one or more sequences of reference nucleotides whose composition is known, and from measurements of current drops produced during one or several passages of these sequence of reference nucleotides through a nanopore sequencer.

In a step 520, measurements of current drop amplitude that are produced as the sequence of nucleotides to be decoded passes through the nanopore device are obtained.

In a step 530, a calculation is performed for each measurement value and for each nucleotide type among the N nucleotide types, of a piece of reliability information based on the Gaussian probability density function obtained for the considered nucleotide type.

The piece of reliability information for a measurement value yₖ and a nucleotide type i is calculated according to equation Math. 8 from Ci, the mean value of the probability density function, and σi, the standard deviation of the probability density function obtained for nucleotide type i.

In a step 540, a decoded value for the measurement value is obtained by applying a soft decoding with an error correction code to each considered current drop value measured and to the N reliability information obtained for the considered value measured. The error correction code is a turbo code or an LDPC, Low-Density Parity-Check, code.

The decoding is based on a modeling of the current drop measurement produced by the nanopore device as a noisy variable modulated by pulse amplitude modulation with N discrete levels. Each level corresponds to an average value of the Gaussian probability density function of the current drop measurements obtained for a given nucleotide among the N types of nucleotides. The modulated noisy variable is noised by N channels of additive white Gaussian noise corresponding respectively to the Gaussian statistical distributions obtained for the N types of nucleotides.

The number N of nucleotide types is equal for example to 4 and the error correction code is applied to quaternary data (or symbols) coded in a Galois field of order 4.

Steps 520 to 540 are repeated for each measurement of a current drop amplitude produced upon passage of a nucleotide of the sequence of nucleotides to be decoded through the nanopore device.

The quaternary data from the decoding is then converted at the output of block 190 into binary data, as described by reference in FIG. 2.

Results

Monte Carlo simulations were performed to obtain performance curves of the DNA-based data storage chain with nanopore sequencing. For this purpose, we generated random binary sequences and converted them into DNA sequences, each nucleotide being represented by a GF(4) symbol. We considered N different values and coding rates for the LDPC code, and compared them to the results obtained with hard detection.

To evaluate the coding gain achieved with the LDPC code, we considered the sequenced nucleotide error rate (SNER) after the base-calling step with HD (that is, uncoded scheme) and the coded SNER (that is, where the soft samples from the nanopore device are the inputs of the non-binary decoder).

For the experimental values presented in the table in FIG. 3, we obtained a simulated SNER_{HD} of 0.23, which is of the same order as the other reported error rates in nanopore devices [17]. Simulations were performed on 10⁶ sequences of coded nucleotides, that is, a data set comprising both original information and redundant symbols forming parity check blocks. We used ultra-sparse non-binary LDPC codes with sequences of N=48 and 192 and 480 GF(4) symbols, and code rates R=½, ⅔ and ⅚. For all considered codes, all sequential errors were corrected, resulting in a nearly error-free sequencing. To be precise, given the number of simulated nucleotide reads, we were able to guarantee error rates on the order of 10⁻⁹. Considering larger blocks (that is, larger values of N) improves the performance. These simulations specifically model substitution errors and they assume perfect correction of insertion and deletion errors. Furthermore, our simulations also assume error-free DNA synthesis and optimal alignment and reconstruction steps. The results obtained here take into account the values presented in FIG. 4 (experimental conditions described in [26]) for LLR calculations in the soft decoder.

The various contributions presented in this document make it possible to use non-binary LDPC codes and their associated low-complexity soft decoding algorithms to greatly improve error performance. Simulation results obtained with ultra-sparse LDPC arrays show that this coding technique is able to correct all sequencing errors of the hard decoding approach. The number of DNA sequences considered in our simulation can guarantee a near error-free performance (SN ER of the order of 10⁻⁹) if we assume having error-free DNA synthesis and perfect correction of insertions and deletions.

The results obtained demonstrate the practical feasibility of non-binary LDPC codes and decoders in DNA storage applications, with appropriately modeled soft intrinsic information used in combination with an optimized Min-Sum decoder.

According to an embodiment, all or part of the decoding method steps described in this document are implemented by a computer software or program.

The functions and methods described in this document can thus be implemented by software (for example, via software on one or several processors, for execution on a general purpose computer (for example, via execution by one or several processors) to implement a special purpose computer or the like) and/or can be implemented in hardware (for example, using a general purpose computer, one or more application-specific integrated circuits (ASIC), and/or any other equivalent hardware).

The present description thus relates to a computer program or software, capable of being executed by a computing device (for example, a computer) serving as a decoding device, by means of one or several data processors, this program/software having instructions for causing said computing device to execute all or part of the steps of one or more of the methods described herein. These instructions are intended to be stored in a memory of a computing device, loaded and then executed by one or several processors of this computing device so as to cause this computing device to execute the method in question.

This software/program may be coded using any programming language, and may be in the form of source code, object code, or intermediate code between source code and object code, such as in a partially compiled form, or in any other desirable form.

The computing device can be implemented by one or several physically separate machines. The computer device may have the overall architecture of a computer, including the components of such an architecture: data memory (memories), processor(s), communication bus(es), hardware interface(s) for connecting this computer device to a network or other equipment, user interface(s), etc.

In one embodiment, some or all of the decoding method steps described in this document are implemented by a decoding device having means for implementing those steps of that method.

These means may comprise software means (for example instructions of one or more program components) and/or hardware means (for example data memory (memories), processor(s), communication bus, hardware interface(s), etc.).

Means implementing a function or a set of functions may also refer in this document to a software component, a hardware component or a set of hardware and/or software components, able to implement the function or the set of functions, as described below for the means concerned.

The present description also relates to an information medium readable by a data processor, and having instructions of a program as mentioned above.

The information medium may be any hardware means, entity or device, capable of storing the instructions of a program as mentioned above. Usable program storage media include ROM or RAM memories, magnetic storage media such as magnetic disks and tapes, hard drives or optically readable digital data storage media, etc., or any combination thereof.

In some cases, the computer-readable storage medium is not transitory. In other cases, the information medium may be a transient medium (for example, a carrier wave) for the transmission of a signal (electromagnetic, electrical, radio or optical signal) carrying program instructions. This signal can be conveyed via an appropriate transmission medium, wired or wireless: electrical or optical cable, radio or infrared link, or by other means.

An embodiment also relates to a computer program product comprising a computer-readable storage medium having program instructions stored thereon, the program instructions being configured to cause??? the computer device to implement some or all of the steps of one or more of the methods described herein when the program instructions are executed by one or several processors and/or one or several programmable hardware components.

According to one embodiment, all or some of the steps of the decoding method described in this document are implemented by electronic circuitry, programmable or not, specific or not.

### LIST OF REFERENCES CITED

[1] G. M. Church, Y. Gao, and S. Kosuri, “Next-generation digital information storage in DNA,” Science, vol. 337, no. 6102, pp. 1628-1628, 2012.
[2] N. Goldman, P. Bertone, S. Chen, C. Dessimoz, E. M. LeProust, B. Sipos, and E. Birney, “Towards practical, high-capacity, lowmaintenance information storage in synthesized DNA,” Nature, vol. 494, no. 7435, pp. 77-80, February 2013.
[3] https://www.microsoft.com/en-us/research/project/DNA-storage/.
[4] https://bigthink.com/philip-perry/microsoft-plans-to-have-a-DNAbased-computer-by-2020.
[5] L. Conde-Canencia and L. Dolecek, “Nanopore DNA sequencing channel modeling,” in 2018 IEEE International Workshop on Signal Processing Systems (SiPS), October 2018, pp. 258-262.
[6] R. N. Grass, R. Heckel, M. Puddu, D. Paunescu, and W. J. Stark, “Robust chemical preservation of digital information on DNA in silica with error-correcting codes,” Angewandte Chemie International Edition, vol. 54, no. 8, pp. 2552-2555.
[7] W. Wan et al., “Error removal in microchip-synthesized DNA using immobilized muts.” Nucleic Acids Res., vol. 42(12), July 2014.
[8] H. M. Kiah, G. J. Puleo, and O. Milenkovic, “Codes for DNA sequence profiles,” IEEE Transactions on Information Theory, vol. 62, no. 6, pp. 3125-3146, June 2016.
[9] F. Sala, R. Gabrys, C. Schoeny, and L. Dolecek, “Three novel combinatorial theorems for the insertion/deletion channel,” in 2015 IEEE International Symposium on Information Theory (ISIT), June 2015, pp. 2702-2706.
[10] F. Sala, R. Gabrys, C. Schoeny, K. Mazooji, and L. Dolecek, “Exact sequence reconstruction for insertion-correcting codes,” in 2016 IEEE Int. Symp. on Inf. Theory (ISIT), July 2016, pp. 615-619.
[11] R. Heckel, I. Shomorony, K. Ramchandran, and D. N. C. Tse, “Fundamental limits of DNA storage systems,” CoRR, vol. abs/1705.04732, 2017. [Online]. Available: http://arxiv.org/abs/1705.04732
[12] M. G. Ross, C. Russ, M. Costello, A. Hollinger, N. J. Lennon, R. Hegarty, C. Nusbaum, and D. B. Jaffe, “Characterizing and measuring bias in sequence data,” Genome Biology, vol. 14, no. 5, p. R51, May 2013.
[13] F. Sala, R. Gabrys, C. Schoeny, and L. Dolecek, “Exact reconstruction from insertions in synchronization codes,” IEEE Transactions on Information Theory, vol. 63, no. 4, pp. 2428-2445, April 2017.
[14] R. Gabrys, H. M. Kiah, and O. Milenkovic, “Asymmetric Lee distance codes for DNA-based storage,” IEEE Transactions on Information Theory, vol. 63, no. 8, pp. 4982-4995, August 2017.
[15] R. Gabrys, E. Yaakobi, and O. Milenkovic, “Codes in the Damerau distance for DNA storage,” CoRR, vol. abs/1601.06885, 2016. [Online]. Available: http://arxiv.org/abs/1601.06885
[16] C. Schoeny, F. Sala, and L. Dolecek, “Novel combinatorial coding results for DNA sequencing and data storage,” IEEE Asilomar Conference on Signals, Systems, and Computers, vol. abs/1801.04882, October 2017.
[17] C. R. O'Donnel, H. Wang, and W. B. Dunbar, “Error analysis of idealized nanopore sequencing,” Electrophoresis, vol. 34(15), pp. 2137-2144, August 2013.
[18] C. Schoeny, A. Wachter-Zeh, R. Gabrys, and E. Yaakobi, “Codes correcting a burst of deletions or insertions,” IEEE Transactions on Information Theory, vol. 63, no. 4, pp. 1971-1985, April 2017.
[19] C. Poulliat, M. Fossorier, and D. Declercq, “Design of regular (2,dc)-LDPC codes over GF(q) using their binary images,” IEEE Trans. Commun., vol. 56, no. 10, pp. 1626-1635, October 2008.
[20] X.-Y. Hu and E. Eleftheriou, “Binary representation of cycle Tanner graph GF(2b) codes,” in IEEE Int. Conf. Commun. ICC′2004. Paris, France, June 2004.
[21] L. Zeng, L. Lan, Y. Tai, S. Song, S. Lin, and K. Abdel-Ghaffar, “Transactions papers—constructions of nonbinary quasi-cyclic LDPC codes: A finite field approach,” Communications, IEEE Transactions on, vol. 56, no. 4, pp. 545-554, April 2008.
[22] R. Peng and R. Chen, “Design of nonbinary quasi-cyclic LDPC cycle codes,” in Information Theory Workshop. Tahoe City, USA, September 2007, pp. 13-18.
[23] A. Venkiah, D. Declercq, and C. Poulliat, “Design of cages with a randomized progressive edge growth algorithm” IEEE Commun. Letters, vol. 12(4), pp. 301-303, April 2008.
[24] I. Wataru, I. Hiroshi, and K. Yoshikazu, “A general method for introducing a series of mutations into cloned DNA using the polymerase chain reaction,” Gene, vol. 102, no. 1, pp. 67-70, 1991.
[25] R. Higuchi, B. Krummel, and R. Saiki, “A general method of in vitro preparation and specific mutagenesis of DNA fragments: study of protein and DNA interactions,” Nucleic Acids Res., no. 16, pp. 7351-67, August 1988.
[26] J. Feng, K. Liu, R. D. Bulushev, S. Khlybov, D. Dumcenco, A. Kis, and A. Radenovic, “Identification of single nucleotides in MoS2 nanopores,” Nature Nanotechnology, vol. 10, pp. 1070-1078, December 2015.
[27] A. Voicila, D. Declercq, F. Verdier, M. Fossorier, and P. Urard, “Low complexity, low memory EMS algorithm for non-binary LDPC codes,” in IEEE Intern. Conf. on Commun., ICC'2007. Glasgow, England, June 2007.
[28] J. Zhao, F. Zarkeshvari, and A. H. Banihashemi, “On implementation of min-sum algorithm and its modifications for decoding LDPC codes,” IEEE Trans. Commun., vol. 53, no. 4, pp. 549-554, April 2005.
[29] D. Declercq and M. Fossorier, “Decoding algorithms for nonbinary LDPC codes over GF(q),” IEEE Trans. Comm., vol. 55, no. 4, pp. 633-643, April 2007.
[30] V. Savin, “Min-max decoding for non binary LDPC codes,” in Proc. IEEE Int. Symp. Information Theory, ISIT'2008. Toronto, Canada, July 2008.
[31] H. Wymeersch, H. Steendam, and M. Moeneclaey, “Log-domain decoding of LDPC codes over GF(q),” in IEEE Intern. Conf. on Commun., ICC'2004. Paris, France, June 2004, pp. 772-776.
[32] E. Boutillon and L. Conde-Canencia, “Bubble check: a simplified algorithm for elementary check node processing in extended min-sum non-binary LDPC decoders,” Electronics Letters, vol. 46, no. 9, pp. 633-634, April 2010.

1. A method for decoding a sequence of binary data encoded by a sequence of nucleotides to be decoded comprising B types of DNA nucleotides, B being an integer equal to 2, 3 or 4, the decoding method comprising
obtaining, for each type of nucleotide of the B types of nucleotides, a probability density function, the probability density functions being obtained from measurements of current drops produced during at least one passage of at least one sequence of reference nucleotides through a nanopore sequencer;
obtaining measurements of current drops (y₁, y₂, . . . yₖ) produced when the sequence of nucleotides to be decoded passes through the nanopore sequencer;
calculating, for at least one measurement value and for each type of nucleotide among the B types of nucleotides, a piece of reliability information (λ^{k}(i)^{L}) based on the probability density function obtained for the type of nucleotide considered;
obtaining, for each considered measurement value, a decoded value identifying a type of nucleotide from the B types of DNA nucleotides by applying a soft decoding algorithm with an error correction code to the current drop measurement and to the B pieces of reliability information obtained for the considered measurement value.

2. The method according to claim 1, wherein the probability density function is a Gaussian probability density function and the soft decoding algorithm is based on modeling the current drop measurement produced by the nanopore sequencer as a noisy variable modulated by pulse-amplitude modulation with B discrete levels, each level corresponding to an average value of the probability density function obtained for a given type of nucleotide, the modulated noisy variable being made noisy by B channels of additive white Gaussian noise corresponding respectively to the statistical distributions obtained for the B types of nucleotides.

3. The method according to claim 1 wherein the error correction code is a turbo code or an LDPC, Low-Density Parity-Check, code decoding algorithm.

4. The method according to claim 1, wherein the soft decoding algorithm is a Min-Sum algorithm for LDPC, Low-Density Parity-Check, codes or a belief propagation algorithm for LDPC codes.

5. The method according to claim 1, wherein the number B of nucleotide types is equal to 4 and the soft decoding algorithm with an error correction code is applied to symbols coded in a Galois Field of order 4, with each symbol in the Galois Field of order 4 corresponding to a nucleotide.

6. The method according to claim 5, wherein the order in which the nucleotide types are associated with the symbols in the Galois Field of order 4 corresponds to the inverse order of the average values of the probability density functions of the current drop amplitudes obtained for the different nucleotide types.

7. The method according to claim 1, wherein the piece of reliability information for a measurement value yₖ and a nucleotide type i is calculated as follows:
$${{λ}^{k} ( i )}^{L} = \frac{{( {y}_{k} - {C}_{i} )}^{2}}{2 {σ}_{i}^{2}} \qquad [ Math \text{.8} ]$$
where Cᵢ is the mean value of the probability density function and σᵢ is the standard deviation of the probability density function obtained for nucleotide type i.

8. A decoding device comprising at least one memory and at least one processor, said at least one memory storing program instructions configured to cause said decoding device to execute the steps of a method according to claim 1 when the program instructions are executed by said at least one processor.

9. A computer program having program instructions for executing the steps of a method according to claim 1 when said program is executed by a computer.

10. A computer-readable recording medium on which is recorded a computer program comprising program instructions for executing the steps of a method according to claim 1 when said program is executed by a computer.

11. A DNA-based data storage system comprising a nanopore sequencer and a decoding device according to claim 8.
